(19) United States
(12) Patent Application Publication
(10) Pub. No.: US 2005/0096520 A1
(43) Pub. Date: May 5, 2005

(54) Extracting device, extracting method, analyzer and analyzing method

(75) Inventors: Yasunori Maekawa, Miki-shi (JP); Kennichi Sawa, Amagasaki-shi (JP); Toshiyuki Sato, Nishinomiya-shi (JP); Seiki Okada, Kobe-shi (JP); Kanako Nagaoka, Kobe-shi (JP); Kaoru Asano, Kobe-shi (JP)

Correspondence Address: BRINKS HOFER GILSON & LIONE, P.O. BOX 10395, CHICAGO, IL, 60610, US

(73) Assignee: Sysmex Corporation

(21) Appl. No.: 10/982,101

(22) Filed: Nov. 4, 2004

(30) Foreign Application Priority Data
Nov. 4, 2003 (JP) 2003-374229

(51) Int. Cl. A61B 5/00

(52) U.S. Cl. 600/365; 604/20; 600/309

(57) ABSTRACT

Extraction devices for extracting analyte through the skin of a living body are disclosed that includes first and second extraction units which are placed on the skin and in which analyte is extracted; electrode unit placed on the skin; and a power source unit for outputting a first current flowing through the electrode unit, the living body, and the first extraction unit, and a second current flowing through the electrode unit, the living body, and the second extraction unit.

This application claims priority under 35 U.S.C. §119 to Japanese Patent Application No. 2003-374229 filed Nov. 4, 2003, the entire content of which is hereby incorporated by reference.

### FIELD OF THE INVENTION

The present invention relates to an extracting device, extracting method, analyzer and analyzing method, and more specifically relates to an extracting device and extracting method for extracting analyte through the skin of a living body, and an analyzer and analyzing method for analyzing the extracted analyte.

### BACKGROUND

In clinical examinations, the presence of a specific material is typically detected in blood obtained by blood collection, and the amount of that material is measured. For example, a diabetic patient manages her own blood sugar by measuring her own blood sugar value, determining her insulin dosage based on this blood sugar value, and determining her food intake restrictions and amount of exercise. Therefore, the diabetic patient must measure her own blood sugar level several times each day. Normally, the blood sugar level is measured using a blood sample collected using a puncturing instrument which is painful and troublesome for the patient. From this perspective, a simple and nonburdening examination which does not require blood collection is in great demand.

In response to this demand, methods for noninvasively extracting analytes from within body tissues without collecting blood, and methods for measuring the amount and concentration of extracted analytes are being developed. An example of such extraction methods is reverse iontophoresis. Reverse iontophoresis is a method for transdermally extracting analyte by applying electrical energy to the skin (for example, U.S. Pat. No. 5,279,543 and WO 96/000110).

The conventional methods and devices mentioned above, however, are painful to the subject when electrical energy is applied to the skin, and in extreme cases the subject may experience a sense of apprehension.

### BRIEF SUMMARY

The scope of the present invention is defined solely by the appended claims, and is not affected to any degree by the statements within this summary.

First extracting device and extracting method embodying features of the present invention are capable of suppressing the pain felt by the subject.

Second extracting device for extracting analyte through the skin of a living body embodying features of the present invention includes first and second extraction units which are placed on the skin and in which analyte is extracted; electrode unit placed on the skin; and a power source unit for outputting a first current flowing through the electrode unit, the living body, and the first extraction unit, and a second current flowing through the electrode unit, the living body, and the second extraction unit.

Third extracting device for extracting analyte through the skin of a living body embodying features of the present invention includes an extracting part having a plurality of extraction units placed on the skin of a living body; extraction energy supplying part for supplying an extraction energy necessary for the extraction of a quantity of analyte necessary for analysis to the plurality of extraction units; and wherein the extraction energy supplying part supplies constant quantity of energy to the respective extraction units regardless of the state of formation of the analyte transmission paths, through which analyte transmits.

Forth extracting method for extracting analyte through the skin of a living body embodying features of the present invention includes placing first and second extraction units in which analyte is extracted, on the skin; placing an electrode unit on the skin; and outputting from a power source unit a first current flowing through the electrode unit, the living body, and the first extraction unit, and a second current flowing through the electrode unit, the living body, and the second extraction unit.

Fifth extracting method for extracting analyte through the skin of a living body embodying features of the present invention includes placing a plurality of extraction units on the skin; supplying extraction energy necessary for the extraction of a quantity of analyte needed for analysis to the plurality of extraction units; and wherein the amount of extraction energy to the respective extraction units are constant regardless of the state of the formation of analyte transmission paths, through which analyte transmits.

### BRIEF DESCRIPTION OF THE DRAWINGS

FIG. 1 is a brief structural view of an embodiment of the extracting device of the present invention;

FIG. 2 is a schematic view of an analyzer provided with the extracting device 1 of FIG. 1;

FIG. 3 is a flow chart illustrating the analysis method used by the analyzer 10.

FIG. 4 is a schematic view of another embodiment of the extracting device of the present invention;

FIG. 5 is a flow chart illustrating the processing performed by the control unit 70 in the extracting device of FIG. 4; and

FIG. 6 is a perspective view of another embodiment of the extraction unit of the present invention.

### DETAILED DESCRIPTION OF THE PREFERRED EMBODIMENTS

The preferred embodiments of the present invention are described hereinafter with reference to the drawings.

The extracting device, extracting method, analyzer, and analyzing method of the present embodiment use the reverse iontophoresis method.

That is, a method is used in the present embodiment, in which analyte within body tissue is noninvasively extracted through the skin (transdermally). Specifically, macropores such as sweat glands and pores and the like, and intercellular micropores are enlarged by the application of electrical energy to the skin so as to form paths for the transmission of analyte through the skin (analyte transmission paths), and analyte is extracted through these paths. Skin includes a horny layer, epidermis, and corium; the tissues below the corium are referred to as living body tissues.

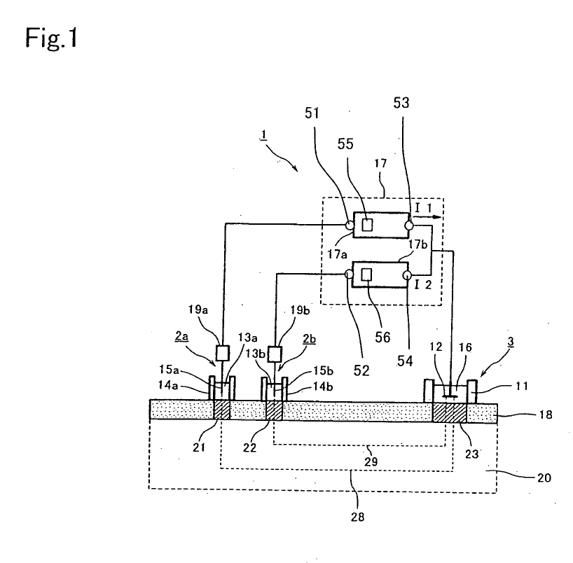

FIG. 1 is a brief structural view of an embodiment of the extracting device 1 of the present invention. The extracting device 1 of this embodiment includes a first extraction unit 2a and second extraction unit 2b positioned on the surface of the skin 18 above living body tissue 20 of a subject, a positive electrode unit 3 similarly positioned on the surface of the skin 18, and a power source unit 17. In the extracting device 1 of this embodiment, two extraction units are provided to reduce the pain felt by the subject.

The first extraction unit 2a is provided with a negative electrode chamber 14a, within which is stored an extraction material collection medium 13a for collecting the extracted analyte, and an extraction electrode 15a (negative electrode) for transmitting electrical energy to the skin is immersed within the extraction material collection medium 13a. Similarly, the second extraction unit 2b is provided with a negative electrode chamber 14b, within which is stored an extraction material collection medium 14b for collecting the extracted analyte, and an extraction electrode 15b (negative electrode) is immersed within the extraction material collection medium 13b.

Furthermore, the positive electrode unit 3 has a positive electrode chamber 11, within which is stored an extraction material collection medium 16, and a positive electrode 12 is immersed within the extraction material collection medium 16.

The power source unit 17 is provided with a first power source 17a and a second power source 17b; in the present embodiment, the first power source 17a and the second power source 17b are both constant-current sources which supply a constant current of 50 μA. The extraction electrode 15a and extraction electrode 15b are respectively connected to the negative terminals 51 and 52 of the first power source 17a and second power source 17b, and the first extraction unit 2a and the second extraction unit 2b can be disengaged by the respective connectors 19a and 19b. Furthermore, the positive electrode 12 of the positive electrode unit 3 is commonly connected to the positive terminals 53 and 54 of the first power source 17a and second power source 17b. In the present embodiment, the first power source 17a and the second power source 17b are provided with voltage limiting units 55 and 56 for limiting the difference on electric potential between the two terminals 51 and 53 and the difference in electric potential between the terminals 52 and 54 so as to not exceed a predetermined value. In the present embodiment, the power sources 17a and 17b are set such that the difference in potential between the two terminals 51 and 53 and the difference in potential between the terminals 52 and 54 do not exceed 10 V. Constant-voltage sources can be used as the first power source 17a and second power source 17b. The constant-voltage sources may be provided with current limiters for limiting the magnitude of the respective output currents so as to not exceed a predetermined value.

Even when a constant-current source is used or a constant-voltage source is used as the first power source 17a and second power source 17b, it is desirable that the difference in potential between the first extraction unit 2a and the positive electrode unit 3, and the difference in potential between the second extraction unit 2b and the positive electrode unit 3 are respectively set so as to be less than 20 V. According to these settings, it is unlikely that the subject will feel any pain.

The extraction electrode 15a of the first extraction unit 2a, the extraction electrode 15b of the second extraction unit 2b and the positive electrode 12 of the positive electrode unit 3 may all have the same construction, or different constructions. Useful materials for the electrodes include Ag, AgCl, carbon, platinum and the like. In the present embodiment, AgCl wire is desirably used as the extraction electrode 15a and extraction electrode 15b, and ring-shaped Ag is desirably used as the positive electrode 12.

Capillaries formed of glass, acrylic or the like may be used as the negative electrode chambers 14a and 14b, and positive electrode chamber 11. In the present embodiment, glass capillaries having internal diameters of φ0.6 mm are used as the negative electrode chambers 14a and 14b, and an acrylic chamber having an internal diameter of φ8 mm is used as the positive electrode chamber 11.

Purified water, ion-conductive aqueous solution (for example, physiological saline solution), hydrogel, ion-conductive hydrogel and the like may be used as the extraction material collection medium 13a, 13b, and 16. Examples of useful ion-conductive hydrogels include gelled materials such as polyacrylate, polyvinyl alcohol, hydroxypropyl cellulose and the like. In the present embodiment, it is desirable that hydroxypropyl cellulose is used as the extraction material collection medium 13a, 13b, and 16.

Furthermore, the first power source 17a and second power source 17b of the power source unit 17 may be direct current sources, alternating current power sources, or a combination of direct current power source and alternating current power source. From the perspective of stability of the amount of extracted analyte, it is desirable that a direct current-type constant-current source is used in the present embodiment.

In the extracting device 1 of the present embodiment, the current (first current) flowing from the first power source 17a of the power source unit 17 flows through a first circuit 28. The first circuit 28 is a circuit from the power source 17a through the positive electrode 12 of the positive electrode unit 3, a region 23 of the skin 18 on which the positive electrode unit 3 is positioned, living body tissue 20, region 21 of the skin 18 on which the first extraction unit 2a is positioned, and the extraction electrode 15a of the first extraction unit 2a, to the first power source 17a. Similarly, the current (second current) flowing from the second power source 17b flows through a second circuit 29. The second circuit is a circuit from the second power source 17b through the positive electrode 12 of the positive electrode unit 3, a region 23 of the skin 18 on which the positive electrode unit 3 is positioned, living body tissue 20, region 22 of the skin 18 on which the second extraction unit 2b is positioned, and the extraction electrode 15b of the second extraction unit 2b, to the second power source 17b.

It is desirable that neither of the currents, that is, the magnitudes of the currents flowing through the first extraction unit 2a and second extraction unit 2b, exceeds 500 μA, and it is highly desirable that the range of the currents is 10 μA to 500 μA. When the current is greater than 10 μA, there is adequate formation of the analyte transmission paths and extraction of the analyte, and insofar as the current does not exceed 500 μA, the subject using the extracting device 1 is unlikely to experience any pain.

In the present embodiment, the magnitude of the current flowing to the first extraction unit 2a and the magnitude of the current flowing to the second extraction unit 2b are substantially the same. Furthermore, the difference in potential between the first extraction unit 2a and the positive electrode unit 3, and the difference in potential between the second extraction unit 2b and the positive electrode unit 3 are substantially the same.

Since the power sources 17a and 17b output current simultaneously in the present embodiment, at a specific moment the power source unit outputs both the first current and second current.

Examples of analyte extracted by the first extracting device 1 of the present embodiment include glucose, lactic acid, ascorbic acid, amino acids, enzyme substrates, drugs and the like.

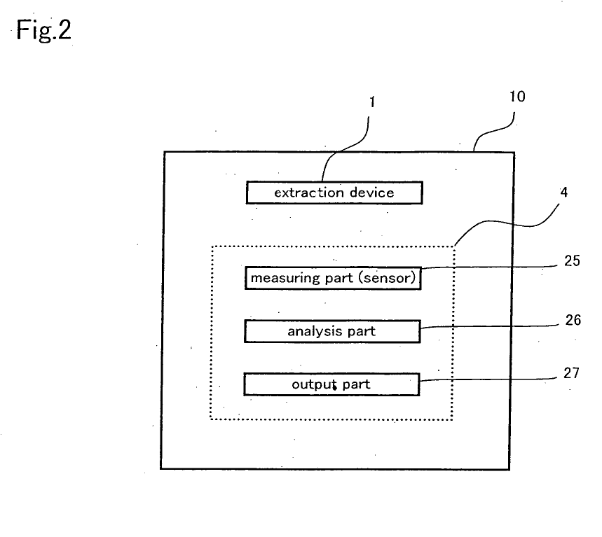

FIG. 2 is a schematic view of an analyzer 10 provided with the previously described extracting device 1. The analyzer 10 is provided with the previously mentioned extracting device 1 and an analysis unit 4. The analysis unit 4 is provided with a measuring part (sensor) 25 for measuring the analyte extracted within the extraction material collection medium 13a and 13b (FIG. 1) and outputting a signal corresponding to the amount of analyte, analyzing part 26 for analyzing the signal output from the measuring part 25, and an output part 27 for outputting (displaying) the analysis result output from the analyzing part 26.

A sensor employing an electrochemical detection method utilized in high-performance liquid chromatography (HPLC) may be used as the measuring part 25. A microcomputer which includes a CPU, ROM, RAM and the like may be used as the analyzing part 26, and a CRT, LCD (liquid crystal display) or the like may be used as the output part 27.

Although the extracting device 1 and analysis unit 4 are separate structures in the present embodiment, the extracting device 1 and analysis unit 4 also may be integrated in a single structure.

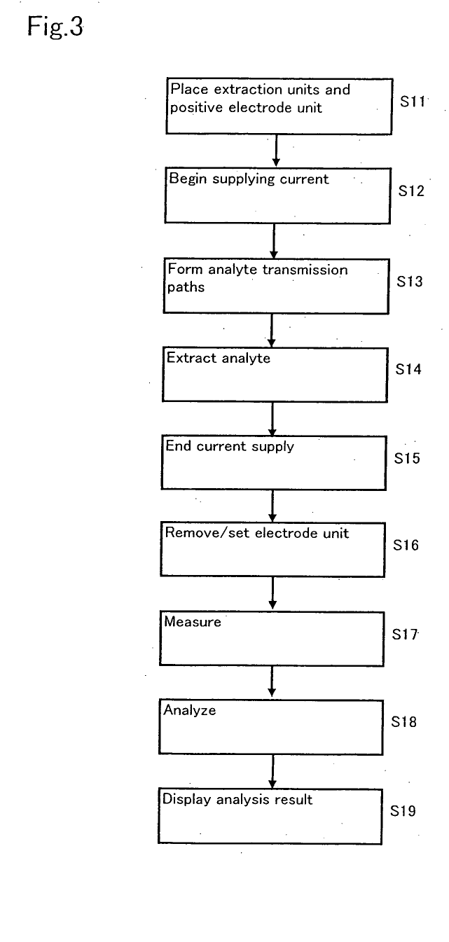

The analysis method used by the analyzer 10 is described below with reference to the flow chart of FIG. 3. First, the subject positions and attaches the first extraction unit 2a, second extraction unit 2b, and positive electrode unit 3 (refer to FIG. 1) to the surface of the skin 18 of the subject (step S11).

The positive electrode 12 of the positive electrode unit 3 is connected to the positive side of the first power source 17a and second power source 17b, the extraction electrode 15a of the first extraction unit 2a is connected to the negative side of the first power source 17a, and the extraction electrode 15b of the second extraction unit 2b is connected to the negative side of the second power source 17b. In this way 50 μA constant currents I1 and I2 are respectively supplied from the first power source 17a and second power source 17b (step 12). The application of the constant current I1 forms a first circuit 28 (FIG. 1) as it flows sequentially from the first power source 17a through the positive electrode 12 of the positive electrode unit 3 through a region 23 of the skin 18, living body tissue 20, region 21 of the skin 18, and the extraction electrode 15a of the first extraction unit 2a, and returns to the first power source 17a. Similarly, the application of the constant current I2 forms a second circuit 29 (FIG. 1) as it flows sequentially from the second power source 17b through the positive electrode 12 of the positive electrode unit 3 through a region 23 of the skin 18, living body tissue 20, region 22 of the skin 18, and the extraction electrode 15b of the second extraction unit 2b, and returns to the second power source 17b. In this way analyte transmission paths are formed in the regions 21 and 22 of the skin 18 (step S13).

When the application of the currents I1 and I2 continues after analyte transmission paths are formed in region 21 and region 22 of the skin 18, ions in the living body tissue 20 migrate through the analyte transmission paths respectively formed in regions 21 and 22 and into the extraction material collection medium 13a and 13b, such that the analyte (glucose) is extracted into the extraction material collection medium 13a and 13b in conjunction with this migration of ions (step S14).

Then the analyte extraction ends by stopping the application of the currents I1 and I2 (step S15).

In steps S13 and S14 of the present embodiment, a 50 μA constant current is respectively supplied for 3 min to the extraction units 2a and 2b regardless of the state of the formation of the analyte transmission paths. In this way a quantity of analyte necessary for analysis can be extracted.

The subject then disconnects the connectors 19a and 19b, and removes the first extraction unit 2a and second extraction unit 2b from the skin 18, and places the extraction units 2a and 2b in the measuring part 25 of the analysis unit 4 (FIG. 2) (step S16).

In the measuring part 25, signals corresponding to the quantities of analyte (glucose) extracted by the extraction material collection medium 13a of the first extraction unit 2a and extracted by the extraction material collection medium 13b of the second extraction unit 2b are output to the analysis part 26 (step S17).

Next, the signals from the measuring unit 25 are analyzed in the analysis part 26, and the analysis result is output to the output part 27 (step S18).

The output part 27 displays the analysis result from the analysis part 26 (step S19), and the analysis by the analyzer 10 ends.

Although the application of the constant current I1 and the application of the constant current I2 is performed simultaneously in steps S12 and S14, these applications also may be performed with a shifted timing. However, simultaneous applications are desirable from the standpoint of reducing the time required for forming the analyte transmission paths and extracting the analyte.

Since current is supplied to the extraction units 2a and 2b by the respective power sources 17a and 17b in the extraction device 1 of the present embodiment, current does not become concentrated in one or another of the extraction units. Accordingly, any sense of pain experienced on the part of the subject is suppressed.

Since the analyte transmission path has not yet formed immediately after starting the application of the constant currents I1 and I2, there is a large resistance in the regions 21 and 22 of the skin 18, and the voltage supplied from the first power source 17a and second power source 17b increases. In the present embodiment, however, since the first power source 17a and second power source 17b are provided with voltage limiting units 55 and 56 for limiting the difference in potential of both terminals of the respective power sources so as to not exceed 10 V, the voltage applied to the regions 21 and 22 of the skin 18 does not exceed 10 V. Accordingly, any sense of pain on the part of the subject can be suppressed by the application of the voltages from the first power source 17a and second power source 17b. Even when the voltage is less than 10 V, analyte transmission paths are ultimately formed in regions 21 and 22 of the skin 18 by the continuous flow of current.

In step S12, an analyte transmission path may be formed first in one or another of the regions 21 and 22 because the conditions in the regions 21 and 22 are not completely identical. In this case, the resistance of the region of the skin in which the analyte transmission path forms is lower, and the current flows more easily. However, since the first power source 17a and second power source 17b are constant current sources in the present embodiment, a current of no more than 50 μA flows to the first extraction unit 2a and second extraction unit 2b, and, accordingly, any feeling of pain on the part of the subject is suppressed.

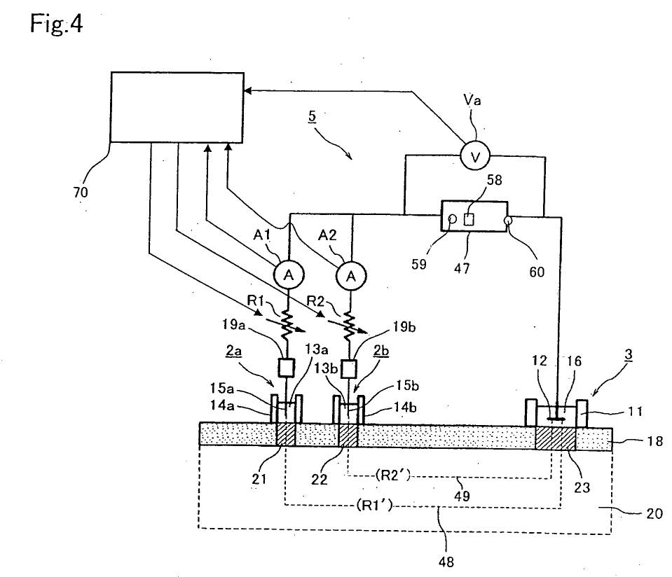

FIG. 4 is a schematic view of the extracting device 5 of another embodiment. The extracting device 5 of this embodiment has a first extraction unit 2a and second extraction unit 2b placed on the surface of the skin 18 above the living body tissue 20 of the subject, an positive electrode unit 3 similarly placed on the surface of the skin 18, and a power source 47. Unlike the embodiment shown in FIG. 1, the present embodiment is provided with only a single power source 47. In the extracting device 5 of the present embodiment, two extraction units are also provided to reduce the sense of pain on the part of the subject.

Similar to the embodiment of FIG. 1, the first extraction unit 2a is provided with a negative electrode chamber 14a, within which is stored an extraction material collection medium 13a for collecting extracted analyte (glucose), and an extraction electrode 15a is immersed within the extraction material collection medium 13a. Likewise, the second extraction unit 2b is provided with a negative electrode chamber 14b, within which is stored an extraction material collection medium 14b for collecting the extracted analyte, and an extraction electrode 15b (negative electrode) is immersed within the extraction material collection medium 13b.

Similar to the embodiment of FIG. 1, the positive electrode unit 3 has a positive electrode chamber 11, within which is stored an extraction material collection medium 16, and an positive electrode 12 is immersed within the extraction material collection medium 16. In the present embodiment, it is also desirable that hydroxypropyl cellulose is used as the extraction material collection medium 13a and 13b.

In the present embodiment, the power source 47 is a constant current source, which outputs a constant current of 100 μA, that is, double the output of the embodiment shown in FIG. 1, since it must supply current to two extraction units 2a and 2b. Furthermore, the power source 47 is provided with a voltage limiter 58 for ensuring that the difference in potential between the two terminals 59 and 60 does not exceed a predetermined value. In the present embodiment, the power source 47 is set such that the difference in potential between the two terminals 59 and 60 does not exceed 10 V. A constant voltage source provided with a current limiter for ensuring that the magnitude of the output current does not exceed a predetermined value may be used as the power source 47.

In the present embodiment, the extraction electrode 15a of the first extraction unit 2a is connected to a variable resistor R1, which is connected to an ammeter A1, and the ammeter A1 is connected to the negative side (terminal 59) of the power source 47. Similarly, the extraction electrode 15b of the second extraction unit 2b is connected to a variable resistor R2, which is connected to a ammeter A2, and the ammeter A2 is connected to the negative side (terminal 59) of the power source 47. Furthermore, in the present embodiment, a voltmeter Va, which measures the difference in potential between the terminals 59 and 60 of the power source 47, is connected between the positive side (terminal 60) and the negative side (terminal 59) of the power source 47. Also in the present embodiment, the first extraction unit 2a and the second extraction unit 2b can be disconnected from the respective connectors 19a and 19b.

The extracting device 5 of the present embodiment shown in FIG. 4 can be combined with the analysis unit 4 of FIG. 2 to form an analyzer.

In the extracting device 5 of the present embodiment, part of the current supplied from the power source 47 flows through a first circuit 48 from the positive electrode 12 of the positive electrode unit 3 through region 23 of the skin 18 on which the positive electrode unit 3 is placed, living body tissue 20, region 21 of the skin 18 on which the first extraction unit 2a is placed, extraction electrode 15a of the first extraction unit 2a, variable resistor R1, and ammeter A1 and returns to the power source 47. Similarly, part of the current supplied from the power source 47 flows through a second circuit 49 from the positive electrode 12 of the positive electrode unit 3 through region 23 of the skin 18 on which the positive electrode unit 3 is placed, living body tissue 20, region 22 of the skin 18 on which the second extraction unit 2b is placed, extraction electrode 15b of the second extraction unit 2b, variable resistor R2, and ammeter A2 and returns to the power source 47.

The extracting device 5 of the present embodiment is provided with a control unit 70 for equalizing the resistance R1′ of the first circuit 48, and resistance R2′ of the second circuit 49. The control unit 70 is provided with a CPU, ROM, and RAM and the like. The control unit 70 is connected to the ammeters A1 and A2, voltmeter Va, and variable resistors R1 and R2. The difference in potential measured by the voltmeter Va and the currents measured by the ammeters A1 and A2 are input to the control unit 70 as digital signals. The control unit 70 executes a process for changing the resistance values of the variable resistors R1 and R2 based on the measured potential difference and current values.

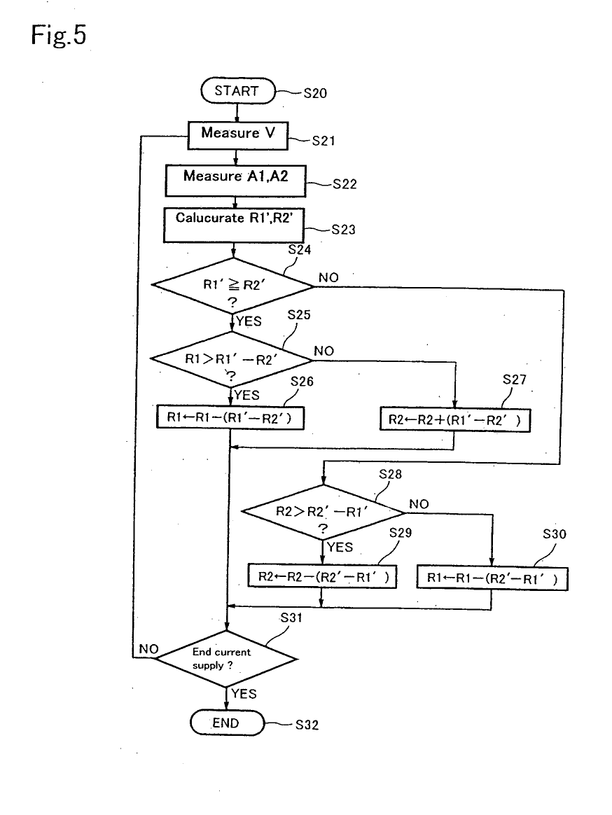

The content of the process executed by the control unit 70 is described below using FIG. 5. As shown in the drawing, the process starts in step S20. First, the potential difference V is measured by the voltmeter Va (step S21), then, the magnitude A1 of the current flowing through the first circuit 48 and the magnitude A2 of the current flowing through the second circuit 49 are measured by the ammeters A1 and A2, respectively (step S22). Next, the resistance value R1′ of the first circuit 48 is calculated by Va/A1, and the resistance value R2′ of the second circuit 49 is calculated by Va/A2 (step S23).

Next, the magnitudes of the resistance value R1′ of the first circuit 48 and the resistance value R2′ of the second circuit 49 are compared (step S24); and when R1′ is greater than R2′, the resistance value R1 of the variable resistor R1 is compared to the value (R1′−R2′) (step S25); and when R1 is greater, the difference between R1 and (R1′−R2′) is substituted for the value R1 (step S26). In this way the resistance value R1′ of the first circuit 48 and the resistance value R2′ of the second circuit 49 are equalized. When it is determined in step S25 that (R1′−R2′) is equal to or greater than R1, the difference between the resistance value R2 of the variable resistor R2 and (R1′−R2′) is substituted for the value R2 (step S27). In this way the resistance value R1′ of the first circuit 48 and the resistance value R2′ of the second circuit 49 are equalized.

When it is determined in step S24 that R2′ is greater than R1′, the resistance value R2 is compared to the value of (R2′−R1′) (step S28); when R2 is greater, the difference between R2 and (R2′−R1′) is substituted for R2 (step S29). In this way the resistance value R1′ of the first circuit 48 and the resistance value R2′ of the second circuit 49 are equalized. When it is determined in step S28 that (R2′−R1′) is equal to or greater than R2, the difference between the resistance value R1 and (R2′−R1′) is substituted for the value R1 (step S30). In this way the resistance value R1′ of the first circuit 48 and the resistance value R2′ of the second circuit 49 are equalized. The resistance value R1′ of the first circuit 48 and the resistance value R2′ of the second circuit 49 are equalized in steps S21 through S30 and, accordingly, the magnitude A1 of the current flowing through the first circuit 48 and the magnitude oA2 of the current flowing through the second circuit 49 are also equalized. This process suppresses any sense of pain perceived by the subject caused by the magnitude of the current flowing in only one path.

Finally, a determination is made as to whether or not the current supply is ending (step S31); when the current application is not ending, the process returns to step S21, and the previously described process is repeated. When it is determined in step S31 that the current application is ending, the process ends in step S32. By repeating steps S21 through S31 in this way, the magnitudes of the currents flowing through the first circuit 48 and second circuit 49 can be normally maintained so as to be equal, thereby suppressing any sensation of pain a subject may feel while forming the analyte transmission paths in the skin 18, and while transdermally extracting the analyte in the living body tissue.

In steps S21 through S31 of the present embodiment, a 50 μA constant current is respectively supplied to the extraction units 2a and 2b regardless of the state of formation of the analyte transmission paths. Accordingly, a quantity of analyte required for analysis can be extracted.

Since the extracting device 5 of the present embodiment distributes the current from the power source 47 to the two extraction units 2a and 2b, the current flow does not become concentrated in only one extraction unit. Therefore, any sensation of pain on the part of the subject is suppressed.

Although a number of variable resistors are used as R1 and R2 in the embodiment of FIG. 4, one resistor may be a fixed resistor and one resistor may be a variable resistor.

The above two embodiments have been described in terms of two extraction unit, however, three or more extraction units may be provided. When three or more extraction units are provided the total quantity of extracted analyte is increased, thus making analysis easier.

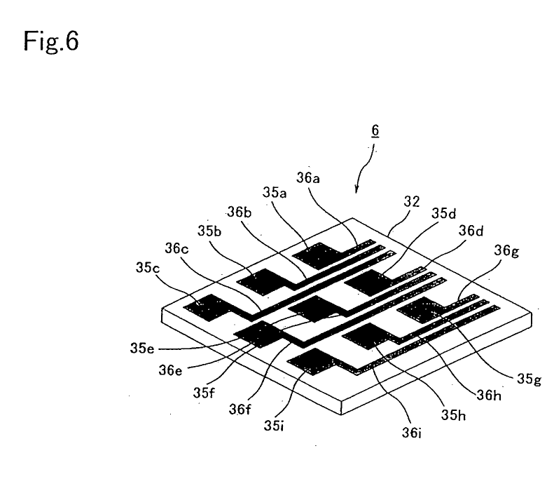

FIG. 6 is a perspective view of an extraction unit 6 of another embodiment of the present invention. The extraction unit 6 of the present embodiment has a single, square-shaped, unified unit 32 which is formed by integratedly combining nine individual extraction units, and nine extraction electrodes 35a, 35b,.35i are arranged in a matrix on the top surface of the unified unit 32. The respective extraction electrodes 35a through 35i are detachably connected to nine constant current sources not shown in the drawing through the respective leads 36a through 36i on the unified unit 32. These nine power sources respectively output a 50 μA constant current, and are respectively provided with voltage limiters to prevent the voltage from exceeding 10 V. In the present embodiment, the unified unit 32 adheres the extraction electrodes 35a through 35i with gel, which functions as an extraction material collection medium; the surface of the unified unit 32 on the side opposite (reverse side of the drawing sheet) the side provided with the extraction electrodes 35a through 35i is adhered to the skin of a subject. The present embodiment also uses constant voltage sources provided with current limiters.

Since current is supplied from nine power sources to the extraction electrodes 35a through 35i in the present embodiment, current does not become concentrated in some of the electrodes, thereby suppressing any sensation of pain on the part of the subject.

Since a constant current source which outputs a 50 μA constant current is used as a power source and is provided with a voltage limiter for limiting the voltage to less than 10 V in the present embodiment, it is unlikely the subject will experience any pain caused by current flowing to some of the electrodes in excess of a predetermined magnitude. Furthermore, since nine individual extraction electrodes 35a through 35i are provided, a large quantity of analyte can be extracted from the living body tissue.

In the extracting devices of all the described embodiments supply energy (current) to each extraction unit for a predetermined unit of time regardless of the condition of the skin, that is, regardless of the condition of the formation of the analyte transmission paths, and the amount of extraction energy necessary for ultimately extracting sufficient analyte for analysis is distributed to a plurality of extraction units. Therefore, the extraction energy does not become concentrated in any part of the skin, and the subject does not experience any pain.

Although the flow of current in the formation of the analyte transmission paths and the extraction of analyte in the present embodiment and all of the above embodiments, the current flow also may be in different directions. Furthermore, analyte transmission path formation and analyte extraction are both performed using the same first extraction unit 2a and extraction unit 2b, the analyte transmission path formation and analyte extraction also may be performed using different extraction units.

Extracting devices exclusively using reverse iontophoresis are shown in each of the above embodiments, however, the present invention is not limited to this arrangement, inasmuch as other usable methods include sonophoresis for extracting analyte in living body tissue by exposing an extraction area of the skin to ultrasonic irradiation to reduce the barrier functionality of the skin and promote passive diffusion, negative pressure suction for extracting analyte in living body tissue by applying negative pressure to an extraction region of the skin to suction analyte, chemical enhancement for enhancing the promotion of transdermal migration of analyte in the extraction region of the skin, and suitable combinations thereof From the perspective of simplifying device construction, it is desirable to use the reverse iontophoresis method so as to use electrical energy as the extraction energy.

In extracting devices 1 and 5 above, an ultrasonic irradiation unit for ultrasonic radiation of the extraction region, a suction unit for suctioning the extraction region under negative pressure, an enhancer adding unit for applying enhancer to the extraction region and the like may be additionally provided. by such addition the analyte extraction amount can be increased so as to make higher precision analysis possible.

It is therefore intended that the foregoing detailed description be regarded as illustrative rather than limiting, and that it be understood that it is the following claims, including all equivalents, that are intended to define the spirit and scope of this invention.

1. An extracting device for extracting analyte through the skin of a living body, comprising:
first and second extraction units which are placed on the skin and in which analyte is extracted;
electrode unit placed on the skin; and
a power source unit for outputting a first current flowing through the electrode unit, the living body, and the first extraction unit, and a second current flowing through the electrode unit, the living body, and the second extraction unit.

2. The extracting device of claim 1, wherein the power source unit comprises first and second power sources, and the first power source outputs the first current, and the second power source outputs the second current.

3. The extracting device of claim 2, wherein the first power source is electrically connected to the first extraction unit and the electrode unit, and the second power source is electrically connected to the second extraction unit and the electrode unit.

4. The extracting device of claim 2, wherein the first power source is a constant current source.

5. The extracting device of claim 4, wherein the constant current power source comprises terminals at both ends, and a voltage limiting unit for limiting the difference in electric potential between the terminals so as to not exceed a predetermined value.

6. The extracting device of claim 2, wherein the first power source is a constant voltage source.

7. The extracting device of claim 6, wherein the constant voltage power source comprises a current limiting unit for limiting the magnitude of the output current so as to not exceed a predetermined value.

8. The extracting device of claim 1, wherein the magnitude of the first current flowing through the first extraction unit and the magnitude of the second current flowing through the second extraction unit are substantially the same.

9. The extracting device of claim 1, wherein the potential difference between the electrode unit and the first extraction unit while the power source unit outputs the first current, and the potential difference between the electrode unit and the second extraction unit while the power source unit outputs the second current are substantially the same.

10. The extracting device of claim 1, wherein the power source unit outputs both the first current and second current at predetermined moments.

11. The extracting device of claim 1, wherein the first current is a current output from the power source unit and sequentially flows through the electrode unit, the living body, and the first extraction unit and returns to the power source unit, and the second current is a current output from the power source unit and sequentially flows through the electrode unit, the living body, and the second extraction unit and returns to the power source unit.

12. The extracting device of claim 1, further comprising
a resistance adjusting unit for adjusting the electrical resistance between the power source unit and the first extraction unit, the resistance adjusting unit being disposed between the power source unit and the first extraction unit; and
a control unit for controlling the resistance adjusting unit such that the magnitude of the first current flowing through the first extraction unit does not exceed a predetermined value.

13. The extracting device of claim 12, further comprising
a current monitor for monitoring the magnitude of the first current, and wherein the control unit controls the resistance adjusting unit based on a monitoring result of the current monitor.

14. The extracting device of claim 1, wherein the first extraction unit comprises an electrode electrically connected to the power source unit, and a collection medium for collecting analyte extracted from living body tissue through the skin.

15. The extracting device of claim 14, wherein the second extraction unit comprises a second electrode electrically connected to the power source unit, and a second collection medium for collecting analyte extracted from living body tissue through the skin.

16. The extracting device of claim 15, wherein the electrode unit comprises a third electrode electrically connected to the power source unit.

17. The extracting device of claim 1, wherein the magnitude of the first current flowing through the first extraction unit and the magnitude of the second current flowing through the second extraction unit do not exceed approximately 500 μA.

18. The extracting device of claim 1, wherein the power source unit outputs the first and second currents such that the potential difference between the electrode unit and the first extraction unit and the potential difference between the electrode unit and the second extraction unit do not exceed approximately 20 V.

19. The extracting device of claim 1, wherein the magnitude of the first current flowing through the first extraction unit does not exceed a predetermined value regardless of the state of formation of analyte transmission paths, through which analyte passes, formed in the skin by the flow of the first current through the living body; and
the magnitude of the second current flowing through the second extraction unit does not exceed a predetermined value regardless of the condition of formation of analyte transmission paths, thorough which analyte passes, formed in the skin by the flow of the second current through the living body.

20. The extracting device of claim 1, wherein the analyte is glucose.

21. An analyzer comprising:
the extracting device of claim 1;
sensor for detecting signals based on the analyte extracted in the first and second extraction units;
analysis unit for analyzing the signals detected by the sensor and obtaining analysis result of analyte; and
an output unit for outputting the analysis result obtained by the analysis unit.

22. An extracting device for extracting an analyte through the skin of a living body, comprising:
an extracting part having a plurality of extraction units placed on the skin of a living body;
extraction energy supplying part for supplying an extraction energy necessary for the extraction of a quantity of analyte necessary for analysis to the plurality of extraction units; and wherein
the extraction energy supplying part supplies constant quantity of energy to the respective extraction units regardless of the state of formation of the analyte transmission paths, through which analyte transmits.

23. The extracting device of claim 22, wherein
the plurality of extraction units have respective extraction electrodes;
the extraction energy supplying part includes a power source for supplying electrical current to the extraction part as the extraction energy; and
the power source supplies a constant current for a predetermined time to the respective extraction electrodes regardless of the state of the formation of the analyte transmission paths.

24. An extracting method for extracting analyte through the skin of a living body, comprising:
placing first and second extraction units in which analyte is extracted, on the skin;
placing an electrode unit on the skin; and
outputting from a power source unit a first current flowing through the electrode unit, the living body, and the first extraction unit, and a second current flowing through the electrode unit, the living body, and the second extraction unit.

25. The extracting method of claim 24, wherein
the power source unit comprises first and second power sources;
the first power source outputs the first current; and
the second power source outputs the second current.

26. The extracting method of claim 24, wherein the magnitude of the first current flowing through the first extraction unit and the magnitude of the second current flowing through the second extraction unit are substantially the same.

27. The extracting method of claim 24, wherein the potential difference between the electrode unit and the first extraction unit while the power source unit outputs the first current, and the potential difference between the electrode unit and the second extraction unit while the power source unit outputs the second current are substantially the same.

28. The extracting method of claim 24, wherein the power source unit outputs both the first current and the second current at predetermined moments.

29. The extracting method of claim 24 further comprising:
adjusting the electrical resistance between the power source unit and the first extraction unit such that the magnitude of the first current flowing through the first extraction unit does not exceed a predetermined value.

30. The extracting method of claim 24, wherein the analyte is glucose.

31. An analyzing method comprising:
the extracting method of claim 24;
detecting signals based on the analyte extracted in the first and second extraction units;
analyzing the detected signals and obtaining an analysis result; and
outputting the obtained analysis result.

32. An extracting method for extracting an analyte through the skin of a living body, comprising:
placing a plurality of extraction units on the skin;
supplying extraction energy necessary for the extraction of a quantity of analyte needed for analysis to the plurality of extraction units; and wherein
the amount of extraction energy to the respective extraction units are constant regardless of the state of the formation of analyte transmission paths, through which analyte transmits.

33. The extracting method of claim 32, wherein the extraction energy is electric energy.
